(19) United States
(12) Patent Application Publication
(10) Pub. No.: US 2017/0369475 A1
(43) Pub. Date: Dec. 28, 2017

(54) Flibanserin Hydrate

(71) Applicant: Sandoz AG, Basel (CH)

(72) Inventors: Andreas Hotter, Kundl (AT); Hannes Lengauer, Kundl (AT); Gerhard Widschwenter, Kundl (AT)

(73) Assignee: Sandoz AG, Basel (CH)

(21) Appl. No.: 15/190,701

(22) Filed: Jun. 23, 2016

(51) Int. Cl. C07D 403/08

(52) U.S. Cl. CPC C07D 403/08; C07B 2200/13

(57) ABSTRACT

The present invention relates to a hydrate of flibanserin, a process for its preparation and to a pharmaceutical composition comprising the hydrate. The invention further relates to the use of said pharmaceutical composition as a medicament in particular for the treatment of hypoactive sexual desire disorder (HSDD).

### FIELD OF THE INVENTION

The present invention relates to a hydrate of flibanserin, a process for its preparation and to a pharmaceutical composition comprising the hydrate. The invention further relates to the use of said pharmaceutical composition as a medicament in particular for the treatment of hypoactive sexual desire disorder (HSDD).

### BACKGROUND OF THE INVENTION

The chemical name of flibanserin is 2H-benzimidazol-2-one-1,3-dihydro-1-[2-[4-[3-(trifluoromethyl)phenyl]-1-piperazinyl]ethyl]. Flibanserin can be represented by the chemical structure according to Formula A

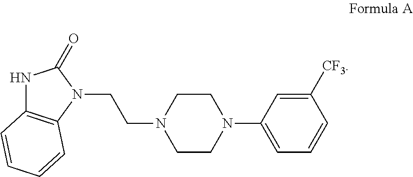 [SMILES: O=c1[nH]c2ccccc2n1CCN1CCN(c2cccc(C(F)(F)F)c2)CC1]

Marketed in the U.S. under the brand name Addyi®, flibanserin is currently indicated for the treatment of premenopausal women with acquired, generalized hypoactive sexual desire disorder (HSDD) as characterized by low sexual desire that causes marked distress or interpersonal difficulty and is not due to a co-existing medical or psychiatric condition, problems within the relationship or the effects of a medication or other drug substance.

The compound flibanserin is disclosed in form of its hydrochloride salt in WO 93/03016.

WO 03/014079 A1 discloses polymorphs A and B of flibanserin. According to the application polymorph A displays a melting point at 161° C., whereas form B shows a melting point at 120° C., when determined with DSC at a heating rate of 10 K/min. However, only a method for polymorph A production is provided in the application but the application is completely silent on how to obtain form B.

The above mentioned products marketed under the trade name Addyi® contain flibanserin as the anhydrous form A.

Different solid forms of an active pharmaceutical ingredient often possess different physical and chemical properties such as melting point, stability, solubility and dissolution rate. In addition, for example depending on environmental conditions such as humidity and temperature the solid forms can convert into each other. The sudden appearance or disappearance of a solid form of an active pharmaceutical ingredient can pose a problem in process development. Similarly, serious pharmaceutical consequences arise if transformation occurs in a dosage form. For example, a conversion to a less stable polymorphic form and the subsequent degradation thereof may lead to the supply of an insufficient amount of the needed active pharmaceutical ingredient. Also, for example the uptake of humidity may lead to a different polymorphic form having a different solubility or dissolution rate.

Also processing or handling of the active pharmaceutical ingredient during the formulation process may be improved by choosing the right solid form. A different morphology i.e. a different particle shape of a solid form of a drug substance can influence the pharmaceutical performance with regards to milling behavior, filterability, flowability, bulk density, compressibility, tableting behavior or dissolution properties and therefore plays a big role in pharmaceutical drug development.

Thus, an objective of the present invention is the provision of another form of flibanserin which increases the repertoire of materials available for a formulation scientist and may allow for the development of more customized formulations such as extended release formulations. In particular it is an objective of the present invention to provide a stable physical form of flibanserin, having improved physical properties over the polymorphic forms of flibanserin known in the prior art, in particular form A, such as dissolution rate, solubility, hygroscopicity, morphology, milling behavior, filterability, flowability, bulk density, compressibility and/ or tableting behavior. In particular, an objective of the present invention is the provision of a stable physical form of flibanserin, which does not transform into other forms during processing or upon storage but is stable over a broad range of water activity even at very wet conditions. A further objective of the present invention is the provision of a stable physical form of flibanserin characterized by a morphology, which translates into improved powder properties, milling and dissolution behavior as well as filterability. Another objective of the present invention is the provision of a pharmaceutical composition comprising a physical form of flibanserin having improved physical properties, the composition is characterized by a consistent bioavailability. Finally, an objective of the present invention is the provision of a customized pharmaceutical flibanserin composition providing an extended release profile. A further objective is the provision of a flibanserin formulation in the form of an aqueous suspension intended for oral or injectable use.

### SUMMARY OF THE INVENTION

The present invention solves one or more of the aforementioned objectives by the provision of a flibanserin hydrate. The flibanserin hydrate of the present invention can be characterized by having a powder X-ray diffractogram comprising reflections at 2-theta angles of (19.5±0.2)° and (25.9±0.2)°, when measured at a temperature in the range of from 20 to 30° C. with Cu-Kalpha_{1,2} radiation having a wavelength of 0.15419 nm. The present invention also provides a process for the preparation of said flibanserin hydrate. Due to its advantageous physical properties, the flibanserin hydrate of the present invention is especially suitable for the preparation of a pharmaceutical composition. Hence, the invention further relates to a pharmaceutical composition comprising flibanserin hydrate and one or more pharmaceutically acceptable excipients and to the use of said pharmaceutical composition as a medicament, in particular for the treatment of hypoactive sexual desire disorder (HSDD).

### Abbreviations

PXRD powder X-ray diffractogram
DSC differential scanning calorimetry
TGA thermogravimetric analyses
GMS gravimetric moisture sorption
RH relative humidity
w- % weight percent

### Definitions

As used herein the term “room temperature” refers to a temperature in the range of from 20 to 30° C.

As used herein, the term “measured at a temperature in the range of from 20 to 30° C.” refers to a measurement under standard conditions. Typically, standard conditions mean a temperature in the range of from 20 to 30° C., i.e. at room temperature. Standard conditions can mean a temperature of about 22° C. Standard conditions can also mean a temperature of about 25° C. Typically, standard conditions can additionally mean a measurement under 20-80% relative humidity, preferably 30-70% relative humidity, more preferably 40-60% relative humidity and most preferably 50% relative humidity.

The term “reflection” with regards to powder X-ray diffraction as used herein, means peaks in an X-ray diffractogram, which are caused at certain diffraction angles (Bragg angles) by constructive interference from X-rays scattered by parallel planes of atoms in solid material, which are distributed in an ordered and repetitive pattern in a long-range positional order. Such a solid material is classified as crystalline material, whereas amorphous material is defined as solid material, which lacks long-range order and only displays short-range order, thus resulting in broad scattering. According to literature, long-range order e.g. extends over approximately 103 to 1020 atoms, whereas short-range order is over a few atoms only (see “Fundamentals of Powder Diffraction and Structural Characterization of Materials” by Vitalij K Pecharsky and Peter Y. Zavalij, Kluwer Academic Publishers, 2003, page 3).

The term “essentially the same” with reference to PXRD means that variabilities in peak positions and relative intensities of the peaks are to be taken into account. For example, a typical precision of the 2-Theta values is in the range of ±0.2° 2-Theta, preferably in the range of ±0.1° 2-Theta. Thus, a diffraction peak that usually appears at 19.5° 2-Theta for example can appear between 19.3° and 19.7° 2-Theta, preferably between 19.4° and 19.6° 2-Theta on most X-ray diffractometers under standard conditions. Furthermore, one skilled in the art will appreciate that relative peak intensities will show inter-apparatus variability as well as variability due to degree of crystallinity, preferred orientation, sample preparation and other factors known to those skilled in the art and should be taken as qualitative measure only.

The term “physical form” as used herein refers to any crystalline and amorphous phase of a substance.

The term “polymorph A” as used herein refers to the crystalline anhydrous form of flibanserin disclosed in WO 03/014079 Al which is characterized by having a PXRD comprising reflections at 2-Theta angles of (15.5±0.2)°, (19.1±0.2)°, (20.1±0.2)°, (22.6±0.2)° and (24.6±0.2)°, when measured at room temperature with Cu-Kalpha_{1,2} radiation having a wavelength of 0.15419 nm.

The term “polymorph” as used herein and as generally understood by the skilled person refers to different crystalline forms of the same molecular entity. Therefore, due to their different chemical compositions, solvates and hydrates are not included in the definition of polymorphism but are rather designated “pseudopolymorphs” instead.

The terms “hydrate” and “hemihydrate” as used herein, refer to crystalline solids were either water is cooperated in or accommodated by the crystal structure e.g. is part of the crystal structure or entrapped into the crystal (water inclusions), or were water is adsorbed on the surface or absorbed in disordered regions of the crystal. Thereby, water can be present in a stoichiometric or non-stoichiometric amount.

A “stoichiometric hydrate” according to the present invention is characterized by possessing a constant mole ratio of flibanserin and water over a range of different water activities. For example, flibanserin hemihydrate of the present invention displays a constant mole ratio of flibanserin and water of 1.0: 0.4 to 0.6, preferably of 1.0: 0.5, when measured with GMS in the range of from 0 to 95% RH.

A “non-stoichiometric” hydrate according to the present invention is characterized in that the mole ratio of flibanserin and water varies continuously as a function of water activity. For example, a non-stoichiometric hydrate of flibanserin displays a variation in the water content of more than 0.5 mole equivalents, when measured with GMS in the range of from 0 to 95% RH.

The terms “stable” or “storage stable” as used herein, mean that the physical form of a compound, e.g., flibanserin, does not change, i.e. does not convert into another physical form when stored for 36 months at long-term condition (25° C./60% RH) or for 6 months at accelerated condition (40° C./75% RH).

A “predetermined amount” as used herein with regards to a certain crystalline form e.g. flibanserin hydrate of the present invention refers to the initial amount of said crystalline form used for the preparation of a pharmaceutical composition.

The term “effective amount” as used herein with regards to a certain crystalline form e.g. flibanserin hydrate of the present invention encompasses an amount of said crystalline form, which causes the desired therapeutic and/or prophylactic effect.

As used herein, the terms “essentially pure” or “substantially pure” with reference to flibanserin hydrate, means that flibanserin hydrate includes less than about 20%, preferably less than about 10%, more preferably less than about 5%, even more preferably less than about 3% and most preferably less than about 1% by weight of any other physical form of flibanserin, in particular polymorph A of WO 03/014079 A1.

As used herein the term “plates” or “plate-like” with regards to particle shape refers to flat, tabular crystals which have similar breadth and width.

The term “laths” as used herein with regards to particle shape refers to elongated, thin and blade-like crystals.

When talking about “flake-like” crystals or “flakes” the present invention means thin, flat crystals that have similar breadth and width and which are thinner than plates.

The term “non-hygroscopic” as used herein refers to a compound which shows a water uptake of at most 0.5 w- % in the sorption cycle when measured with gravimetric moisture sorption at a relative humidity in the range of from 0 to 95% and a temperature of 25.0±0.1° C., based on the weight of the compound,

The term “about” as used herein means within 5%, more typically within 1% and most typically within 0.5% of the indicated value or range.

As used herein, the term “mother liquor” refers to the solution remaining after crystallization of a solid.

### BRIEF DESCRIPTION OF THE DRAWINGS

FIG. 1: illustrates a representative PXRD of crystalline flibanserin hydrate of the present invention. The x-axis shows the scattering angle in °2-theta, the y-axis shows the intensity of the scattered X-ray beam in counts of detected photons.

FIG. 2: illustrates a comparison of representative PXRDs of crystalline flibanserin hydrate of the present invention (bottom) and polymorph A of flibanserin of WO 03/014079 A1 (top). The x-axis shows the scattering angle in °2-theta. The intensities of the two PXRDs were first adapted so that the most intensive reflections of the two forms had similar intensities and then the PXRD of polymorph A was shifted along the y-axis to separate the PXRDs for clarity reason. The y-axis is therefore arbitrary and was not labeled.

FIG. 3: illustrates a representative DSC curve of flibanserin hydrate of the present invention. The x-axis shows the temperature in degree Celsius (° C.), the y-axis shows the heat flow rate in Watt per gram (W/g) with endothermic peaks going up.

FIG. 4: illustrates a representative TGA curve of flibanserin hydrate of the present invention. The x-axis shows the temperature in degree Celsius (° C.), the y-axis shows the mass (loss) of the sample in weight percent (w- %). FIG. 5: illustrates representative GMS isotherms of flibanserin hydrate of the present invention in the range of from 0 to 95% relative humidity. The x-axis displays the relative humidity in percent (%) measured at a temperature of (25.0±0.1) ° C., the y-axis displays the equilibrium mass change in weight percent (w- %). The sorption cycle is marked by triangles, whereas the desorption cycle is marked by squares. The values are displayed as uncorrected values.

FIG. 6: illustrates a representative photomicrographic image of lath shaped flibanserin hydrate crystals of the present invention under a polarizing light microscope.

FIG. 7: illustrates a photomicrographic image of plate-like (tabular) flibanserin polymorph A crystals (obtained according to the procedure disclosed in WO 03/014079 A1) under a polarizing light microscope.

### DETAILED DESCRIPTION OF THE INVENTION

The present invention relates to flibanserin hydrate.

Flibanserin hydrate can be characterized by the chemical structure according to formula B

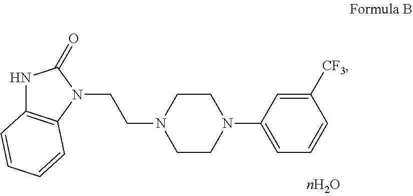

wherein n is at least 0.1, preferably at least 0.2, more preferably at least 0.3, even more preferably at least 0.4, for example at least 0.5. In a preferred embodiment, n is not more than 6.0, preferably not more than 4.0, more preferably not more than 2.0 even more preferably not more than 1.0 and most preferably not more than 0.6. In particular, n is between 0.1 and 6.0, 0.1 and 4.0, 0.1 and 2.0, 0.1 and 1.0, 0,1 and 0.8, 0.1 and 0.6, 0.1 and 0.5, 0.1 and 0.4, 0.1 and 0.3, 0.2 and 6.0, 0.2 and 4.0, 0.2 and 2.0, 0.2 and 1.0, 0.2 and 0.8, 0.2 and 0.6, 0.2 and 0.5, 0.2 and 0.4, 0.3 and 6.0, 0.3 and 4.0, 0.3 and 2.0, 0.3 and 1.0, 0.3 and 0.8, 0.3 and 0.6, 0.3 and 0.5, 0.4 and 6.0, 0.4 and 4.0, 0.4 and 2.0, 0.4 and 1.0, 0.4 and 0.8, 0.4 and 0.6, 0.5 and 6.0, 0.5 and 4.0, 0.5 and 2.0; 0.5 and 1.0, 0.5 and 0.8, 0.6 and 6.0, 0.6 and 4.0; 0.6 and 2.0, 0.6 and 1.0 or 0.6 and 0.8.

Once isolated, the hydrate of the present invention shows high stability against conversion into other forms and against thermodynamically degradation and preserves a constant water content over the whole range of relative humidity. For example, flibanserin hydrate shows neither significant water uptake nor loss, when subjected to atmospheres having a relative humidity in the range of from 0 to 95% (see FIG. 5 herein). While stability against moisture could have been expected for a hydrate, the fact that flibanserin hydrate of the present invention preserves its crystal water even when subjected to dry conditions as low as 0% relative humidity is indeed surprising because hydrates very often tend to lose their water (dehydrate) under such extreme conditions.

Solubility and dissolution rate of flibanserin hydrate of the present invention are naturally different from those of polymorph A of WO 03/014079 A1 and allow for the formulation of more customized formulations such as extended release formulations, and aqueous suspensions either for oral or for injectable use.

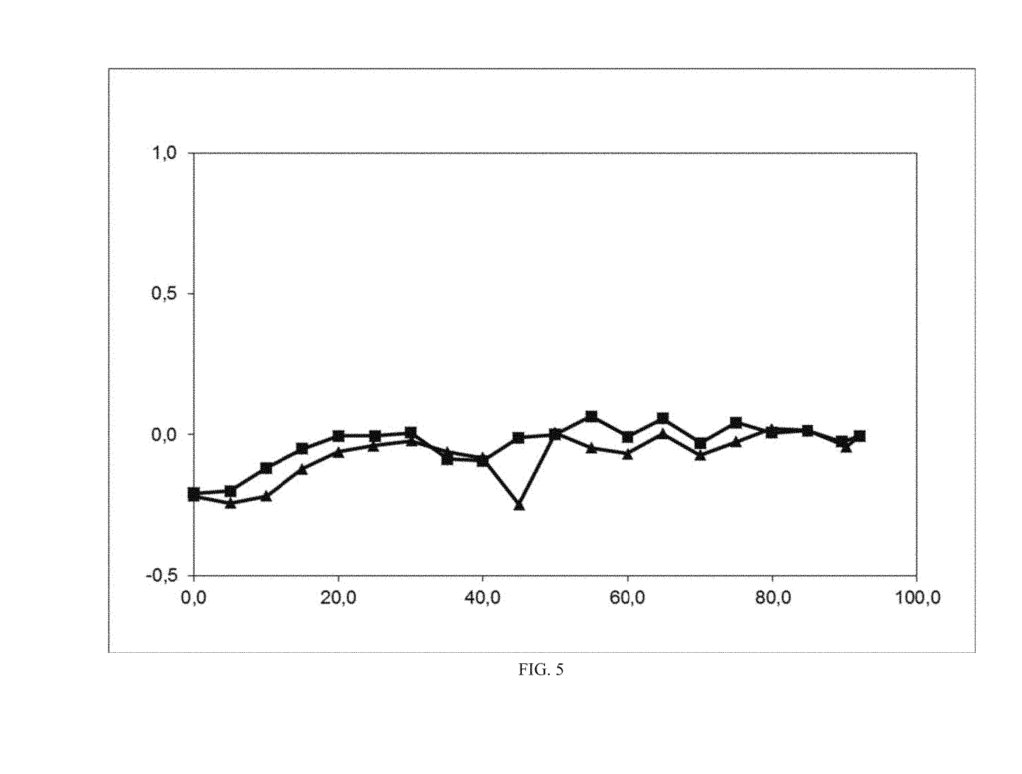

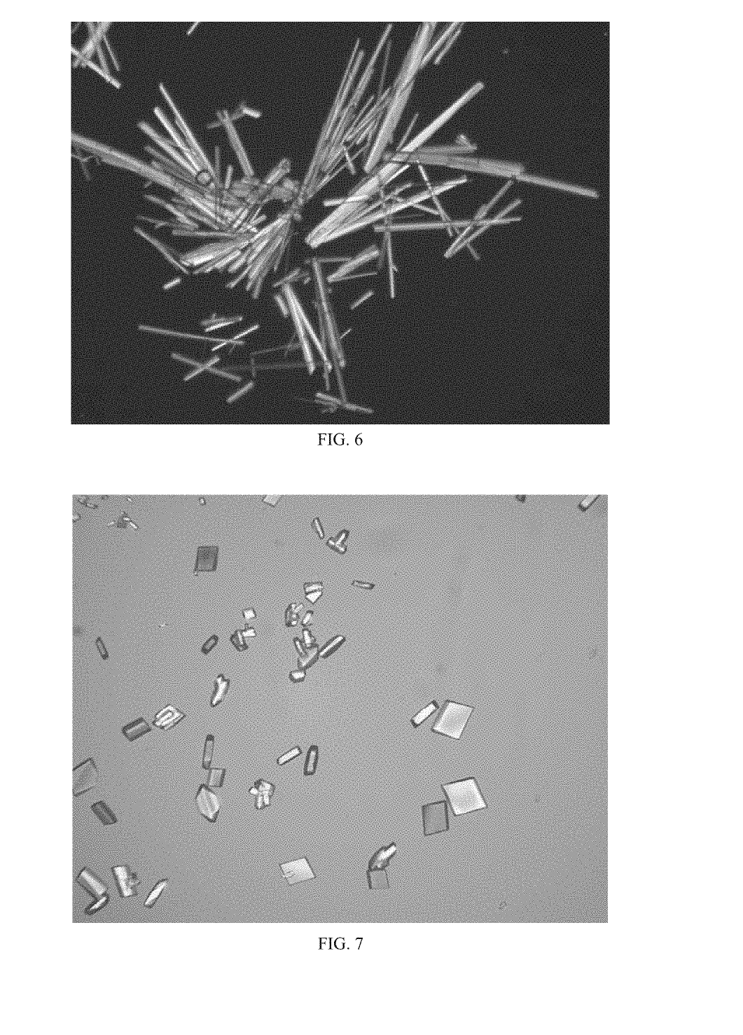

In addition, due to their unique morphology (shape), the flibanserin hydrate crystals of the present invention show favorable processing properties with regards to milling behavior, filterability, flowability, bulk density, compressibility, tableting behavior or dissolution properties. In contrast to the plate-like crystals of polymorph A (see FIG. 7 herein), the lath shaped flibanserin hydrate crystals (see FIG. 6 herein) of the present invention in addition allow the production of material with a greater spectrum of different particle size distributions (PSDs). E.g. the laths obtained according to the procedure disclosed in example 1 herein can be easily broken by means of milling, thus providing the formulation scientist with material having a broad repertoire of different PSDs. Also the morphology of the lath shaped crystals of flibanserin hydrate may be altered by milling processes. For example the laths may be broken to flake-like or plate-like particles, which again possess different powder properties and increase the repertoire of materials available for a formulation scientist with regards to particle shape.

Alternatively, flibanserin hydrate of the present invention may also be used as intermediate for the production of polymorph A of WO 03/014079 A1 having homogenous particle size distribution. Advantageously, upon heating the hydrate of the present invention can be dehydrated, whereupon the crystals break apart to yield small crystallites of polymorph A having homogenous particle size distribution. This process avoids harsh mechanical stress occurring during milling processes, which may lead to undesired crystal defects. The dehydration process may also lead to polymorph A particles having lath shaped morphology.

The invention is described below in further detail by embodiments, without being limited thereto.

Flibanserin hydrate of the present invention may be characterized by analytical methods well known in the field of the pharmaceutical industry for characterizing solids. Such methods comprise but are not limited to PXRD, DSC, TGA, GMS and (polarizing) light microscopy. The hydrate of the present invention may be characterized by one of the aforementioned analytical methods or by combining two or more of them. In particular, flibanserin hydrate may be characterized by any one of the following embodiments or by combining two or more of the following embodiments.

Hence, in a first aspect the present invention relates to flibanserin hydrate characterized by having a PXRD comprising reflections at 2-Theta angles of:
(19.5±0.2)° and (25.9±0.2)°; or
(19.5±0.2)°, (22.9±0.2)° and (25.9±0.2)°; or
(12.0±0.2)°, (19.5±0.2)°, (22.9±0.2)° and (25.9±0.2)°; or
(12.0±0.2)°, (14.0±0.2)°, (19.5±0.2)°, (22.9±0.2)° and (25.9±0.2)°; or
(6.0±0.2)°, (12.0±0.2)°, (14.0±0.2)°, (19.5±0.2)°, (22.9±0.2)° and (25.9±0.2)°; when measured at a temperature in the range of from 20 to 30° C. with Cu-Kalpha_{1,2} radiation having a wavelength of 0.15419 nm.

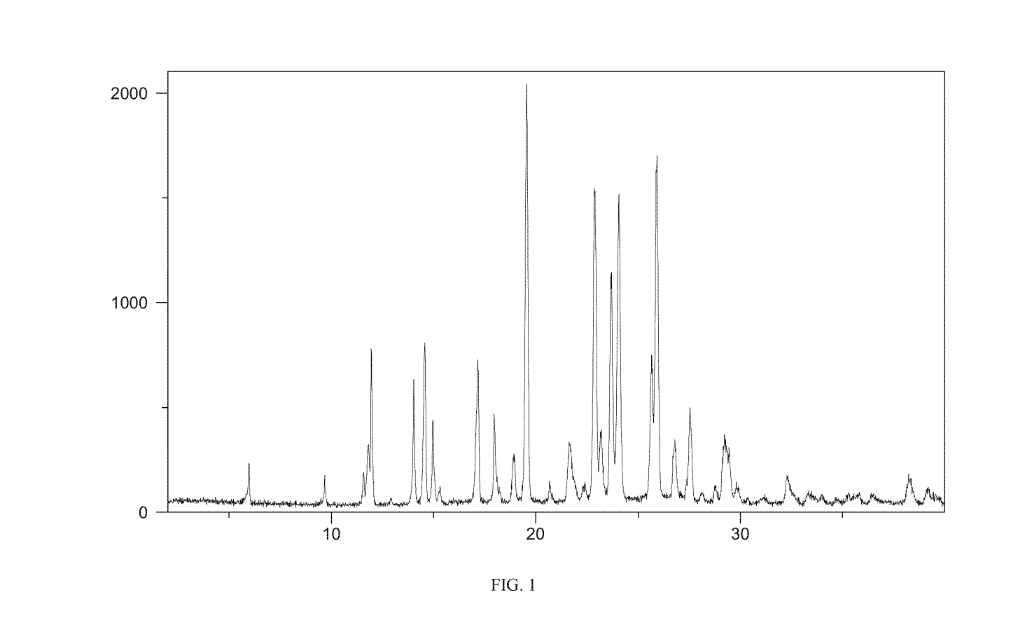

In another embodiment, the present invention relates to flibanserin hydrate characterized by having a PXRD essentially the same as shown in FIG. 1 of the present invention, when measured at a temperature in the range of from 20 to 30° C. with Cu-Kalpha_{1,2} radiation having a wavelength of 0.15419 nm.

The PXRD of flibanserin hydrate of the present invention can be clearly distinguished from the

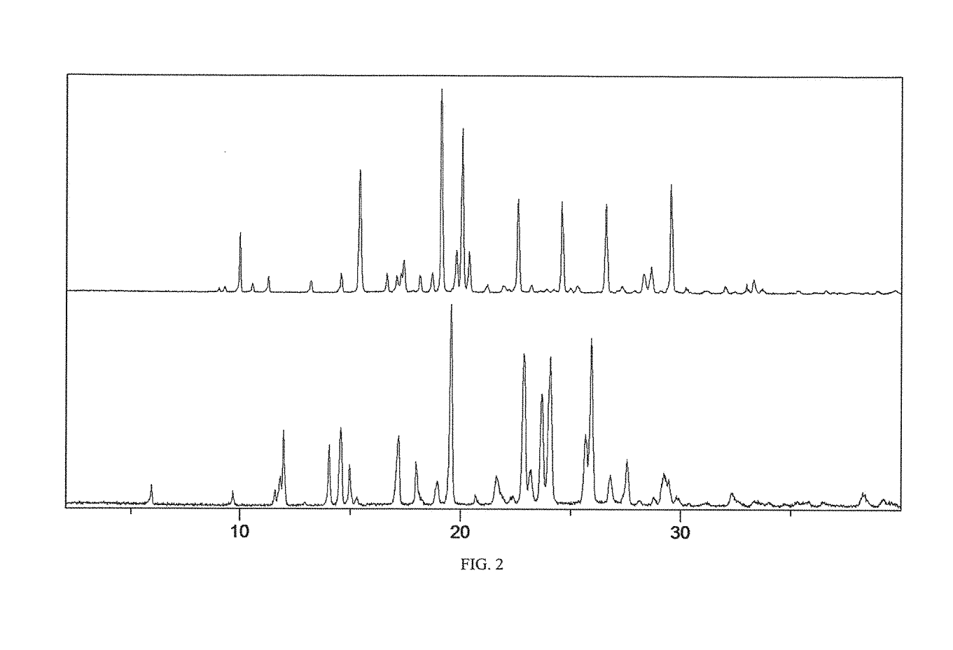

PXRD of polymorph A of WO 03/014079 A1 (see also PXRD overlay displayed in FIG. 2 herein). For instance, flibanserin hydrate shows a reflection at 2-Theta angles of (6.0±0.2)°, (12.0±0.2)°, (14.0±0.2)° and (25.9±0.2)°, whereas polymorph A shows no reflection in the same ranges. On the other hand, polymorph A for example shows a reflection at 2-Theta angles of (20.0±0.2)° and (24.6±0.2)°, which are missing in the diffractogram of flibanserin hydrate.

In a further preferred embodiment, the present invention relates to flibanserin hydrate, characterized by showing an endothermic peak, preferably a first endothermic peak at a temperature in the range of from about 80 to 130° C., preferably from about 90 to 120° C. and most preferably from about 90 to 110° C., when measured with DSC at a heating rate of 10 K/min. Preferably, the first endothermic peak has an onset temperature in the range of from about 80 to 100° C., preferably from about 85 to 95° C. In a particular embodiment, the present invention relates to flibanserin hydrate, characterized by showing an endothermic peak, preferably a first endothermic peak at a temperature of about (108±2) ° C., said peak having an onset temperature of about (91±2) ° C., when measured with DSC at a heating rate of 10 K/min. Preferably, the first endothermic signal is due to the release of one or more solvent(s), most preferably the one or more solvent(s) is water. Most preferably, the first endothermic signal is caused by dehydration.

In another embodiment, the present invention relates to flibanserin hydrate characterized by showing a mass loss in the range of from 1.8 to 2.8 w- %, preferably from 2.0 to 2.6 w- % and most preferably from 2.2 to 2.4 w- % based on the weight of the hydrate, when measured with TGA at a temperature in the range of from 25 to 130° C. and a heating rate of 10 K/min. Preferably, the mass loss is caused by the release of one or more solvent(s), most preferably the one or more solvent(s) is water. Most preferably, the mass loss is caused by dehydration.

In yet another embodiment, the present invention relates to flibanserin hydrate characterized by showing a mass change of not more than 0.5 w- %, preferably of not more than 0.4 w- %, more preferably of not more than 0.3 w- % and most preferably of not more than 0.2-w%, based on the weight of the hydrate, when measured with GMS at a relative humidity in the range of from 0 to 95% and a temperature of (25.0±0.1) ° C.

The non-hygroscopic behavior of flibanserin hydrate of the present invention is highly appreciated as the physicochemical properties of the hydrate are preserved regardless the relative humidity of the surrounding atmosphere, which facilitates the manufacturing process as well as storage of pharmaceutical products comprising the hydrate. For example, there is no need for expensive packaging material which protects the drug product comprising flibanserin hydrate of the present invention from humid atmospheres. Surprisingly, flibanserin hydrate of the present invention is also stable at dry conditions and preserves its crystal structure even at relative humidities as low as about 0%. This is remarkable as hydrates often tend to lose their crystal water at very dry conditions.

In still another embodiment, the present invention relates to flibanserin hydrate characterized by having lath shaped crystals. The lath shaped crystals of flibanserin hydrate are clearly distinguishable from the plate-like polymorph A crystals.

In a further embodiment, the present invention refers to a flibanserin hydrate in the form of a hemihydrate, characterized by a mole ratio of flibanserin and water >1, preferably in the range of from 1.0: 0.4 to 0.6, more preferably the mole ratio of flibanserin and water is 1.0: 0.5.

A preferred flibanserin hemihydrate can be characterized by the chemical structure according to formula B

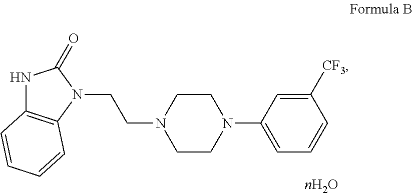

wherein n is <1, preferably in the range of from 0.1 to 0.8, 0.1 to 0.6, 0.1 to 0.4, 0.2 to 0.8, 0.2 to 0.6, 0.2 to 0.4, 0.3 to 0.8, 0.3 to 0.6, more preferably 0.4 to 0.6, most preferably n is 0.5.

It is worthwhile mentioning, that flibanserin hydrate as disclosed herein was not accessible via a routine screening process but first crystals were only obtained by serendipity from an oily residue containing some droplets of water as inclusions. Almost discarded, said oily residue was observed to contain some crystals which have grown upon storage. These crystals were identified as first flibanserin hydrate crystals ever. Once said crystals were available, they were used as seeds in a solvent crystallization procedure. Only since then, flibanserin hydrate could be routinely produced by the process disclosed hereinafter.

Hence, in a further aspect, the present invention relates to a process for the preparation of flibanserin hydrate of the present invention said process comprising the steps of:
(a) providing a solution comprising flibanserin, 1,4-dioxane and water,
(b) maintaining the solution provided in step (a) at a temperature in the range of from 35 to 45° C.,
(c) adding flibanserin hydrate seed crystals as defined herein to the solution in step (b),
(d) optionally separating at least a part of the crystals obtained in step (c) from their mother liquor,
(e) optionally drying the crystals obtained in step (d).

Flibanserin can be prepared according to the teaching of WO 03/014079 A1. The applied solid to form is not critical because the starting material is completely dissolved in step (a) of the above defined process. In a first step, a solution of flibanserin in aqueous 1,4-dioxane is provided. Preferably, flibanserin starting material is first dissolved in 1,4-dioxane alone before water is added. More preferably, the dissolution step is accelerated by heating the flibanserin/1,4-dioxane mixture to a temperature in the range of from about 40° C. to about reflux temperature, more preferably the mixture is warmed to a temperature in the range of from about 40 to 80° C., most preferably from about 40 to 60° C. The flibanserin concentration of the thereby obtained 1,4-dioxane solution preferably is in the range of from about 40 to 80 g/L, more preferably from about 50 to 70 g/L. Subsequently, water is added to the solution, whereat the addition is performed in such a manner that the temperature of the initial solution does not drop below 40 ° C. For example, water is added dropwise to the 1,4-dioxane solution. The 1,4-dioxane/water ratio of the final mixture is approximately 1: 1 (volume:volume), a slight excess of water may be used though. However, it is crucial that after complete water addition the solution is still clear and that no turbidity, which indicates the spontaneous nucleation and subsequent crystallization of flibanserin, occurs. Once the water addition is complete, the obtained solution may be filtrated.

Importantly, in a subsequent step, the solution obtained in step (a) is tempered at a temperature in the range of from about 35 to 45° C., most preferably the temperature of the solution is adjusted to about 40° C. before flibanserin hydrate seed crystals as defined herein are added. Maintaining the temperature in the range of from about 35 to 45° C., such as 40° C. is key to obtain the desired flibanserin hydrate of the present invention as phase pure form, in particular free of polymorph A.

Seed crystals of flibanserin hydrate as defined hereinabove are added to promote crystallization of flibanserin hydrate. Seed crystals can be prepared according to the specific example 2 disclosed herein. The amount of seed crystals employed may range from about 1 to 20 w- %, preferably from about 1 to 10 w- % and most preferably from about 1 to 5 w- %, based on the weight of applied flibanserin starting material. Once seed crystals have been added the mixture may be further kept at a temperature in the range of from about 35 to 45° C., such as 40° C. without agitation, e.g. without stirring, in order to increase the yield.

The obtained flibanserin hydrate crystals may optionally be separated from their mother liquor by any conventional method such as filtration or centrifugation, most preferably by filtration.

Optionally, the isolated crystals may be washed with a solvent. Preferably, the solvent comprises water and most preferably the solvent is water.

Finally, flibanserin hydrate crystals may optionally be dried at a temperature of about 60° C. or less, preferably of about 40° C. or less, more preferably of about 30° C. or less and most preferably the crystals are dried at room temperature. Drying may be performed for a period in the range of from about 1 to 72 hours, preferably from about 2 to 48 hours, more preferably from about 4 to 24 hours and most preferably from about 6 to 18 hours. Drying may be performed at ambient pressure and/ or under vacuum preferably at about 100 mbar or less, more preferably at about 50 mbar or less and most preferably at about 30 mbar or less, for example at about 20 mbar or less.

In another aspect, the present invention relates to the use of flibanserin hydrate as described hereinabove for the preparation of a pharmaceutical composition.

In a further aspect, the present invention relates to a pharmaceutical composition comprising flibanserin hydrate as defined hereinabove and one or more pharmaceutical acceptable excipient(s). In a preferred embodiment, the present invention relates to a pharmaceutical composition comprising a predetermined amount of flibanserin hydrate as defined hereinabove and one or more pharmaceutical acceptable excipient(s). In yet a further preferred embodiment, the present invention relates to a pharmaceutical composition comprising an effective amount of flibanserin hydrate as defined hereinabove and one or more pharmaceutically acceptable excipient(s). In a preferred embodiment, the effective amount of flibanserin is 100 mg calculated as anhydrous flibanserin. In a further preferred embodiment, the one or more pharmaceutically acceptable excipient(s) is/ are selected from the group consisting of fillers, binders, disintegrants, lubricants, coating agents and colorants. In yet a further preferred embodiment, the one or more pharmaceutically acceptable excipient(s) is/ are selected from the group consisting of lactose monohydrate, microcrystalline cellulose, hypromellose, croscarmellose sodium, magnesium stearate, talc, macrogol, titanium dioxide and iron oxide.

In another preferred embodiment, the pharmaceutical composition of the present invention is a tablet, a capsule or an aqueous suspension, most preferably the pharmaceutical composition is a tablet. Preferably, the aqueous suspension is intended for oral or injectable use.

In one embodiment, the pharmaceutical composition as described hereinabove is preferably administered orally, more preferably once daily, most preferably once per day at bedtime.

In another preferred embodiment, the pharmaceutical composition as defined hereinabove is taken for at least 8 weeks.

The present invention also relates to the pharmaceutical composition as defined hereinabove for use as a medicament.

Finally, the invention relates to the pharmaceutical composition as defined hereinabove for the treatment of hypoactive sexual desire disorder (HSDD). In a preferred embodiment, the invention relates to the pharmaceutical composition as defined hereinabove for the treatment of premenopausal women with acquired, generalized hypoactive sexual desire disorder (HSDD)

The following non-limiting examples are illustrative for the disclosure and shall not limit the scope of the invention.

### EXAMPLES

Powder X-ray Diffraction (PXRD)

PXRD was performed with a PANalytical X′Pert PRO diffractometer equipped with a theta/theta coupled goniometer in transmission geometry, Cu-Kalpha_{1,2} radiation (wavelength 0.15419 nm) with a focusing mirror and a solid state PIXcel detector. Diffractograms were recorded at a tube voltage of 45 kV and a tube current of 40 mA, applying a stepsize of 0.013° 2-Theta with 40 s per step (255 channels) in the angular range of 2° to 40° 2-Theta at ambient conditions. A typical precision of the 2-Theta values is in the range of ±0.2° 2-Theta, preferably in the range of ±0.1° 2-Theta. Thus, the diffraction peak of flibanserin hydrate that appears for example at 19.5° 2-Theta can appear between 19.3° and 19.7° 2-Theta, preferably between 19.4° and 19.6° 2-Theta on most X-ray diffractometers under standard conditions.

A representative powder X-ray diffractogram of flibanserin hydrate is displayed in FIG. 1 and the corresponding reflection list is provided in table 1 hereafter.

TABLE 1
Reflection list and corresponding relative intensities
of flibanserin hydrate between 2.0 and 30.0° 2-Theta

| Angle [±0.2° 2-Theta] | Relative Intensity [%] |
|---|---|
| 6.0 | 10 |
| 9.7 | 7 |
| 11.6 | 8 |
| 11.8 | 14 |
| 12.0 | 37 |
| 14.0 | 30 |
| 14.6 | 39 |
| 15.0 | 21 |
| 15.3 | 4 |
| 17.2 | 34 |
| 18.0 | 20 |
| 18.9 | 11 |
| 19.5 | 100 |
| 20.7 | 5 |
| 21.7 | 15 |
| 22.4 | 5 |
| 22.9 | 75 |
| 23.2 | 18 |
| 23.7 | 54 |
| 24.1 | 75 |
| 25.7 | 36 |
| 25.9 | 84 |
| 26.8 | 15 |
| 27.5 | 23 |
| 28.1 | 3 |
| 28.8 | 4 |
| 29.2 | 16 |
| 29.8 | 4 |

Differential Scanning Calorimetry (DSC)

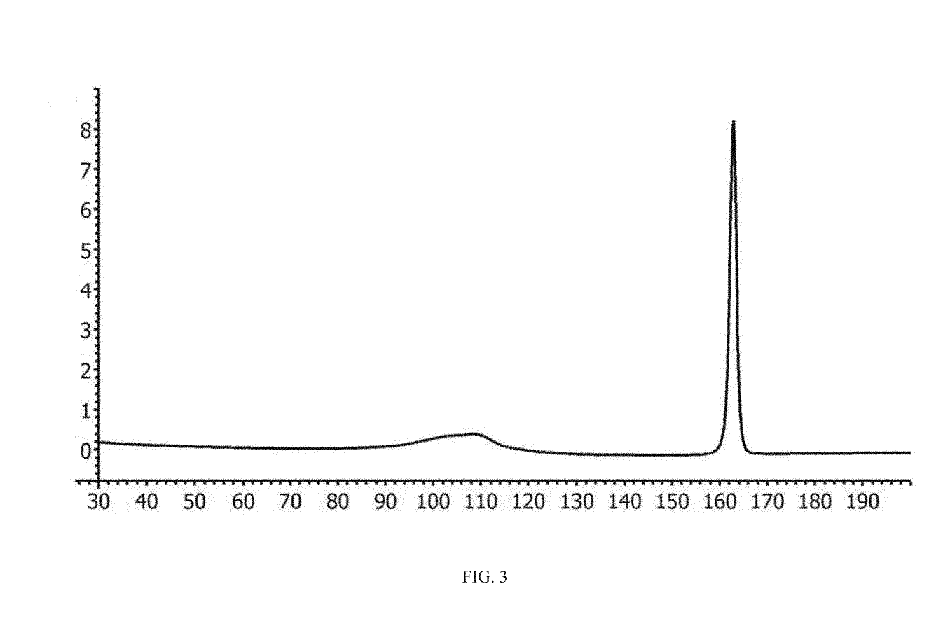

DSC was performed on a Mettler Polymer DSC R instrument. The sample (3.45 mg) was heated in a 40 microL aluminium pan with a pierced aluminium lid from 25 to 200° C. at a rate of 10° K/min. Nitrogen (purge rate 50 mL/min) was used as purge gas. A representative DSC curve is displayed in FIG. 3 hereinafter and shows a first broad endotherm with an onset temperature of about 91° C. and a peak temperature of about 108° C., which is due to the loss of water (dehydration). After dehydration of flibanserin hydrate, polymorph A of WO 03/014079 A1 is present, which shows a melting endotherm with an onset temperature of about 161° C. and a peak temperature of about 162° C.

Thermogravimetric Analysis (TGA)/Coulometric Karl Fischer Titration

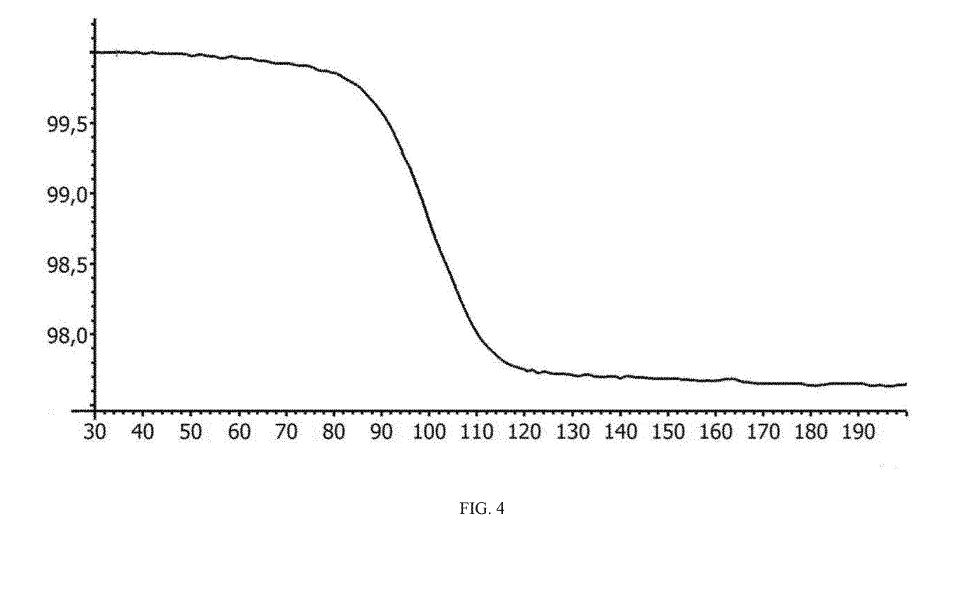

TGA was performed on a Mettler TGA/DSC 1 instrument. Flibanserin hydrate (5.91 mg) was heated in a 100 microL aluminium pan closed with an aluminium lid from 25 to 200° C. at a rate of 10 K/min. The lid was automatically pierced at the beginning of the measurement. Nitrogen (purge rate 50 mL/min) was used as purge gas. A representative TGA curve is displayed in FIG. 4 hereinafter and shows a step from about 45 to 130° C., which is due to the loss of water (dehydration). The mass during the step was determined to be about 2.3% which corresponds to 0.5 mole of water per mole flibanserin. The water value was also confirmed by Coulometric Karl-Fischer titration performed on a Metrohm 831 KF Coulometer connected to a KF Thermoprep 832 oven, which was heated to 130° C. for the measurement.

Gravimetric Moisture Sorption (GMS)

Moisture sorption isotherms were recorded with an SPSx-1 μ moisture sorption analyzer (ProUmid, Ulm). The measurement cycle was started at ambient relative humidity (RH) of 30%. RH was then decreased to 5% in 5% steps, followed by a further decrease to 3% and to 0%. Afterwards RH was increased from 0% to 95% in a sorption cycle and subsequently decreased to 0% in a desorption cycle each in 5% steps. Finally, RH was increased to ambient relative humidity of 30% in 5% steps.

The time per step was set to a minimum of 2 hours and a maximum of 6 hours. If an equilibrium condition with a constant mass of ±0.01% within 1 hour was reached before the maximum time for all examined samples the sequential humidity step was applied before the maximum time of 6 hours. If no equilibrium was achieved the consecutive humidity step was applied after the maximum time of 6 hours. The temperature was 25±0.1° C.

FIG. 5 shows the equilibrium mass changes (delta m in weight% on the y-axis) of flibanserin hydrate during the sorption cycle (marked by triangles) from 0% to 95% RH, as well as during the desorption cycle (marked by squares) from 95 to 0% RH (on the x-axis). The mass difference between 0 and 95% RH was only 0.2 weight % and no significant hysteresis was observed. Therefore, flibanserin hydrate of the present invention can be assigned as being non-hygroscopic. The lacking hysteresis between the sorption and the desorption isotherms indicates that no structural changes have appeared during the experiment. This assumption was strengthened by the fact that the sample still showed the same PXRD after the experiment.

### Example 1:

### Preparation of Flibanserin Hydrate—Best Mode

Flibanserin (2.0 g, polymorph A prepared according to the teaching disclosed in WO 03/014079 A1) was dissolved in 1,4-dioxane (30 mL) upon heating. Water (20 mL) was added dropwise to the hot solution. The obtained solution was filtered while hot and cooled to 40° C. before additional water (6 mL) and flibanserin hydrate seed crystals (0.1 g, prepared according to example 2 hereinafter) were added. Subsequently, additional water (5 mL) was added to the mixture, whereupon crystallization was observed. The mixture was kept at 40° C. without mechanical agitation before the obtained crystals were collected by filtration, washed with water (20 mL) and sucked dry on the filter.

Yield: 0.9 g (42% of Theory)

### Example 2:

### Preparation of Flibanserin Hydrate Seed Crystals

Flibanserin (100 mg, polymorph A prepared according to the teaching disclosed in WO 03/014079 A1) were dissolved in aqueous THF (90 volume %, 2 mL) upon heating. The solution was filtrated while hot and kept at room temperature without agitation for 3 days in a sealed vial. As no crystallization was observed the cap was removed from the vial and the solvent was allowed to evaporate slowly overnight, whereat an oily residue containing droplets of water as inclusions was obtained. The vial was left standing at room temperature for another night, whereupon crystals of flibanserin hydrate were obtained. The so obtained material was used as seed crystals in example 1 herein.

Yield: 87 mg (85% of Theory)

The subjects and preferred embodiments of the present invention can be illustrated by the following items:
1. Flibanserin hydrate.
2. The flibanserin hydrate of item 1 characterized by the chemical structure according to formula B

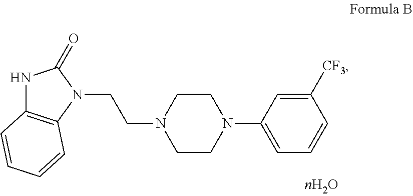

wherein n is <1, preferably 0.1 to 0.8, 0.1 to 0.6, 0.1 to 0.4, 0.2 to 0.8, 0.2 to 0.6, 0.2 to 0.4, 0.3 to 0.8 or 0.3 to 0.6, more preferably 0.4 to 0.6, most preferably n is 0.5.
3. The flibanserin hydrate of item 2 wherein n is 0.1 to 0.8, 0.1 to 0.6, 0.1 to 0.4, 0.2 to 0.8, 0.2 to 0.6, 0.2 to 0.4, 0.3 to 0.8, 0.3 to 0.6, 0.4 to 0.6 or n is 0.5.
4. The flibanserin hydrate according to anyone of the preceding items characterized by having a powder X-ray diffractogram comprising reflections at 2-theta angles of (19.5 0.2)° and (25.9±0.2)°, when measured at a temperature in the range of from 20 to 30 ° C. with Cu-Kalphai_{1,2} radiation having a wavelength of 0.15419 nm.
5. The flibanserin hydrate of item 4 characterized by having a powder X-ray diffractogram comprising an additional reflection at a 2-Theta angle of (22.9±0.2)°, when measured at a temperature in the range of from 20 to 30° C. with Cu-Kalphai_{1,2} radiation having a wavelength of 0.15419 nm.
6. The flibanserin hydrate of item 4 or 5 characterized by having a powder X-ray diffractogram comprising an additional reflection at one or more 2-Theta angles selected from the group of (6.0±0.2)°, (12.0±0.2)° and (14.0±0.2)°, when measured at a temperature in the range of from 20 to 30° C. with Cu-Kalphai_{1,2} radiation having a wavelength of 0.15419 nm.
7. Use of the flibanserin hydrate according to any one of the preceding items for the preparation of a pharmaceutical composition.
8. Pharmaceutical composition comprising the flibanserin hydrate as defined in any one of items 1 to 6 and one or more pharmaceutically acceptable excipient(s).
9. The pharmaceutical composition of item 8, wherein the one or more pharmaceutically acceptable excipient(s) is/ are selected from the group of fillers, binders, disintegrants, lubricants, coating agents and colorants.
10. The pharmaceutical composition of item 8 or 9 wherein the one or more pharmaceutically acceptable excipient(s) is/are selected from the group of lactose monohydrate, microcrystalline cellulose, hypromellose, croscarmellose sodium, magnesium stearate, talc, macrogol, titanium dioxide and iron oxide.
11. The pharmaceutical composition according to any one of items 8 to 10, which is a tablet ora capsule.
12. The flibanserin hydrate of anyone of items 1 to 6 or the pharmaceutical composition according to any one of items 8 to 11 for use as a medicament.
13. The flibanserin hydrate of anyone of items 1 to 6 or the pharmaceutical composition according to any one of items 8 to 11 for use in the treatment of hypoactive sexual desire disorder (HSDD).
14. Process for the preparation of the flibanserin hydrate of any one of items 1 to 6 comprising the steps of:
   (a) providing a solution comprising flibanserin, 1,4-dioxane and water,
   (b) maintaining the solution provided in step (a) at a temperature in the range of from 35 to 45° C.,
   (c) adding flibanserin hydrate as defined in any one of items 1 to 6 as seed crystals to the solution in step (b),
   (d) optionally, separating at least a part of the crystals obtained in step (c) from their mother liquor. flibanserin hydrate crystals obtained in step c)
15. The process of item 14 further comprising drying the crystals obtained in step (c) or (d).

1. Flibanserin hydrate characterized by the chemical structure according to formula B 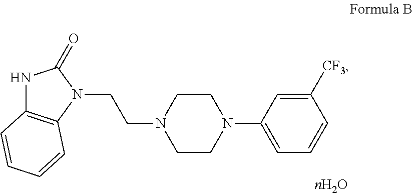
wherein n is <1 and
having a powder X-ray diffractogram comprising reflections at 2-theta angles of (19.5±0.2)° and (25.9±0.2)°, when measured at a temperature in the range of from 20 to 30° C. with Cu-Kalpha_{1,2} radiation having a wavelength of 0.15419 nm.

2. (canceled).

3. The flibanserin hydrate of claim 1 wherein n is 0.1 to 0.8, 0.1 to 0.6, 0.1 to 0.4, 0.2 to 0.8, 0.2 to 0.6, 0.2 to 0.4, 0.3 to 0.8, 0.3 to 0.6, 0.4 to 0.6 or n is 0.5.

4. (canceled).

5. The flibanserin hydrate of claim1 characterized by having a powder X-ray diffractogram comprising an additional reflection at a 2-Theta angle of (22.9±0.2)°, when measured at a temperature in the range of from 20 to 30° C. with Cu-Kalpha_{1,2} radiation having a wavelength of 0.15419 nm.

6. The flibanserin hydrate of claim 1 characterized by having a powder X-ray diffractogram comprising an additional reflection at one or more 2-Theta angles selected from the group of (6.0±0.2)°, (12.0±0.2)° and (14.0±0.2)°, when measured at a temperature in the range of from 20 to 30° C. with Cu-Kalpha_{1,2} radiation having a wavelength of 0.15419 nm.

7. Pharmaceutical composition comprising the flibanserin hydrate as defined in claim 1 and one or more pharmaceutically acceptable excipient(s).

8. The pharmaceutical composition of claim 7, wherein the one or more pharmaceutically acceptable excipient(s) is/are selected from the group of fillers, binders, disintegrants, lubricants, coating agents and colorants.

9. The pharmaceutical composition of claim 7, wherein the one or more pharmaceutically acceptable excipient(s) is/are selected from the group of lactose monohydrate, microcrystalline cellulose, hypromellose, croscarmellose sodium, magnesium stearate, talc, macrogol, titanium dioxide and iron oxide.

10. The pharmaceutical composition according to claim 7, which is a tablet or a capsule.

11. A method for treating hypoactive sexual desire disorder (HSDD)
comprising administering to a subject in need thereof a therapeutically effective amount of the compound according to claim 1.

12. Process for the preparation of the flibanserin hydrate of claim 1 comprising the steps of:
(a) providing a solution comprising flibanserin, 1,4-dioxane and water,
(b) maintaining the solution provided in step (a) at a temperature in the range of from 35 to 45° C.,
(c) adding flibanserin hydrate as defined in claim 1 as seed crystals to the solution in step (b),
(d) optionally, separating at least a part of the crystals obtained in step (c) from their mother liquor. flibanserin hydrate crystals obtained in step c).

13. The process of claim 12 further comprising drying the crystals obtained in step (c) or (d).
